(19) United States
(12) Patent Application Publication
(10) Pub. No.: US 2017/0007788 A1
(43) Pub. Date: Jan. 12, 2017

(54) Actuator and Nose Piece for a Nasal Inhaler

(71) Applicant: 3M INNOVATIVE PROPERTIES COMPANY, Saint Paul, MN (US)

(72) Inventor: Richard D Brewer, Loughborough (GB)

(21) Appl. No.: 15/114,246

(22) PCT Filed: Jan. 28, 2015
(86) PCT No.: PCT/US2015/013190

§ 371 Date: Jul. 26, 2016

(30) Foreign Application Priority Data
Jan. 31, 2014 (GB) 1401659.6

(51) Int. Cl. A61M 15/08; A61M 15/00

(52) U.S. Cl. CPC A61M 15/08; A61M 15/0068; A61M 15/009; A61M 2207/00; A61M 2205/276

(57) ABSTRACT

The present invention provides an actuator (10) for a nasal inhaler (5). The actuator comprises an elongate body (15) comprising a canister opening (11), a nose piece (30) adapted for nasal delivery of a medicament, and fixing means for fixing the nose piece to the elongate body (15). The nose piece is fixable to the body at an acute angle to the elongate body. A first portion of the nose piece defines an outlet (25) for discharging a medicament from the actuator. The nose piece is adapted and the elongate body is adapted so that when the nose piece is fixed to the elongate body, the nose piece and elongate body cooperate to define a fluid communication pathway from the canister opening to the outlet. The present invention is also directed to an inhaler comprising an actuator as discussed, and a canister.

The present invention relates to actuators for metered dose nasal inhalers, to metered dose nasal inhalers, to methods for producing actuators for such inhalers and to methods of assembling nasal inhalers.

Pressurized metered dose inhalers (pMDI) may be used for delivering medication in the form of aerosols to patients. The route of delivery of the medicament using such inhalers may be oral or nasal.

Such an inhaler commonly comprises a canister containing the medicament aerosol formulation, and an actuator with a delivery passage. The canister contains the aerosol formulation, either as a solution or suspension, in the form of one or more drugs and propellant, and optionally excipients, selected from co-solvents, surfactants, stabilizing substances (for chemical or physical stability) and flavourings. The canister also comprises a metering valve arranged to deliver a metered dose of the medicament on actuation of the inhaler.

The actuator typically comprises a housing, generally made of a plastic material, within which the canister is located. A portion of the canister will usually project above the actuator housing.

In oral inhalers the actuator has a delivery passage in the form of a mouthpiece that is placed in the patient's mouth and through which the medicament passes on being dispensed. The patient places the mouthpiece in their mouth and breathes in, creating an air flow from the actuator through the mouthpiece and into the mouth and lungs. At the same time the patient actuates dispensation of the medicament from the canister. Actuation may occur as a result of inhalation by the patient or the patient may manually actuate the inhaler, for example, by depressing the projecting portion of the canister further into the housing. Nasal actuators operate in a similar fashion, but instead of a mouthpiece the actuator is provided with a nosepiece for delivery of the medicament to the nasal passages. In the case of nasal medicament delivery, there is not a need for the concurrent inhalation of air, however.

U.S. Pat. No. 3,913,842 discloses a spray head or nozzle apparatus adapted to guide aerosol propelled medicaments into various body openings, for example the ear canal.

U.S. Pat. No. 3,361,306 discloses aerosol devices having a metering dispensing valve for discharging from a container a measured amount of a liquid as a mist or vapour, the liquid comprising a liquefied gaseous propellant under pressure, containing a dissolved or suspended medically active ingredient.

WO-A-2005/120617 discloses an adaptation of an oral inhaler device for nasal delivery and more particularly to a method of adapting for nasal delivery a metered dose oral aerosol inhaler device, and a nasal adaptor for an oral metered dose aerosol inhaler device

WO-A-98/031411 discloses an aerosol inhalation device that is manually operated and comprises a holding part for receiving an aerosol container with a valve and outlet tip, an inspiratory part and a member for passage of aerosol.

US-A-2003/0089368 discloses nozzles for aerosol propellant systems, and more particularly aerosolization spray nozzles for metered dose inhalers.

U.S. Pat. No. 3,361,306 discloses an aerosol device for dispensing a liquid containing a medically active ingredient dissolved or suspended therein.

WO-A-99/25407 discloses an actuator for an inhaler for administering medicament by inhalation.

GB-A-2,143,283 discloses applicators for dispensing medicaments from a pressurised dispensing container.

GB-A-2,170,430 discloses improvements relating to spray nozzles, particularly of the kind that are used to dispense a fine spray of liquid.

GB-A-1,021,739 discloses a device for use in inhalation therapy with aerosols.

GB-A-2,366,519 discloses a dispensing apparatus for use with pressurised dispensing containers and, in particular, an apparatus for dispensing orally inhaled medicinal products in aerosol form.

GB-A-2,415,388 discloses a delivery device for products, such as medicaments, and particularly a device for transferring to the portal regions of the respiratory tract of a patient a metered dose of a product contained in a pressurised dispensing container.

Known actuators as discussed above do not, however, take account of the need to accommodate various designs of canister valve. Actuators in the documents listed above do not take account of the need to provide devices to improve the assurance for patients in their use of inhalers, for example, dose indicators nor to improve the comfort and ease of use of inhalers. Furthermore, it may be complex and costly to manufacture actuators to the required tolerances and quality.

In a first aspect, the present invention accordingly provides an actuator for a nasal inhaler, the actuator comprising an elongate body comprising a canister opening for insertion of a canister for supplying a medicament, a nose piece adapted for nasal delivery of the medicament, the nose piece being fixable to the body at an acute angle to the elongate body, a first portion of the nose piece defining an outlet for discharging a medicament from the actuator, and fixing means for fixing the nose piece to the body, wherein the nose piece is adapted and the body is adapted so that when the nose piece is fixed to the body the nose piece and body cooperate to define a fluid communication pathway from the canister opening to the outlet.

The nose piece usually further comprises a second portion of the nose piece defining an orifice through which medicament may pass from the body to the nose piece. When fixed with the body, the orifice will usually be in direct fluid communication (and therefore in contact) with an expansion chamber defined by the elongate body which is in fluid communication with the canister opening. The expansion chamber may comprise a transition chamber.

The first portion of the nose piece defines an outlet for discharging a medicament from the actuator, preferably directly into the nostril of the patient. Consequently, it is preferred if, when in use by a patient, the nose piece is in contact with the patient. Preferably, the nose piece defines a delivery passage portion which comprises and defines the outlet for discharging the medicament from the actuator.

Usually, the actuator comprises a stem socket for receiving the valve stem of the canister. Preferably, the stem socket is comprised in a stem post.

Preferably, the stem post comprises an expansion chamber for receiving at least a portion of a metered dose of the medicament from the canister.

Having a separate nose piece and elongate body is advantageous because it facilitates manufacture of an actuator because each part may be optimised. In particular, a nose piece may be produced with a relatively longer orifice and/or the body with a relatively longer, optional, expansion chamber. The possibility of having a longer orifice allows selection of appropriate characteristics of the spray exiting from the orifice. It is important to optimise the spray characteristics for medicament delivery, and for patient comfort particularly in nasal drug delivery applications. The longer, optional, expansion chamber allows more space to incorporate other components in the actuator (e.g. a dose counter). The longer orifice and/or the longer, optional, expansion chamber are of particular benefit when the spray is directed at an upward angle for intranasal administration.

The nose piece may be adapted to be fixable solely by the action of insertion. In some embodiments additional fixing means such as clips, adhesives and/or welding portions may also be used to fix the delivery passage portion and the body. Welding may be heat, ultrasonic and/or laser welding.

When fixed with the body, the nose piece is preferably angled upwardly (i.e. towards the top, canister end of the actuator) at an acute angle with respect to the long axis of the body, preferably at 85° or less, more preferably at 75° or less or 70° or less, most preferably at about 66°. In an alternative embodiment, the nose piece may be angled downwardly at an acute angle with respect to the long axis of the elongate body, preferably at 85° or less, more preferably at 75° or less or 70° or less, most preferably at about 66°.

The orifice, preferably, comprises a jet portion of predetermined width and predetermined length, that preferably extends from the expansion chamber and/or the transition chamber, if present, to the orifice outlet. The predetermined width may be in the range 0.1 mm to 1.5 mm. The predetermined length may be in the range 0.05 mm to 5 mm, preferably 0.4 mm to 3 mm. The orifice may be generally of any cross sectional shape (e.g. oval, rectangular) but is preferably generally circular.

The fixing means preferably comprises at least one press fit seal which may be a ring and groove press fit seal. The ring portion of a ring and groove press fit seal may be on the nose piece, with the groove situated in the actuator body. Alternatively, the ring may be on the body with the groove on the nose piece. The press fit seal preferably forms an interference fit seal when engaged.

The fixing means may comprise an adhesive portion (where an adhesive has been used to fix the nose piece and body) and/or a welded portion (where a welding process has been used to fix the nose piece and body). The welded portion may be an ultrasonic, laser and/or heat welded portion.

The fixing means may additionally comprise at least one clip.

The fixing means is preferably tamper-proof, or advantageously at least tamper-evident to discourage a patient from separating the nose piece from the actuator body.

Preferably, the body and/or nose piece may further comprise alignment features, for example asymmetric lugs, keying features, cradles, clips or flat surfaces that in combination with other alignment features define a position of alignment and engagement.

The nose piece may be a unitary moulding (i.e. the nose piece may be or is produced by moulding in one piece). This is particularly advantageous because it leads to manufacturing efficiency. Previously, it has been difficult to consistently mould an actuator in one piece, at least partly because of the need for tight tolerances and particularly minimal moulding flash. This has previously been a particular problem in the case of nasal inhalers where there is an acute angle between the stem socket/expansion chamber and delivery passage in the nose piece of the actuator. In the present invention, this problem is addressed by the use of a separate but fixable nose piece (usually of a smaller size than the assembled actuator), which enables tighter tolerances to be achieved in the mould with a significant reduction in flash.

It is advantageous if the elongate body is a unitary moulding (i.e. the body may be or is produced by moulding in one piece).

The elongate body may further comprise a window, preferably an indicator viewing window. The indicator viewing window is particularly useful for display of a dose indication or a dose count if the actuator further comprises a dose indicator or a dose counter.

Preferably, the elongate body further comprises supporting means for supporting a dose indicator or a dose counter. Such supporting means may be, for example wing portions.

In a second aspect, there is provided an inhaler comprising an actuator as discussed in relation to the first aspect, and a canister.

In a preferred aspect, the actuator may be produced by moulding, preferably injection moulding.

In a third aspect, the present invention provides a method of assembling a nasal actuator for an inhaler, the method comprising providing an elongate body comprising a canister opening for insertion of a canister for supplying a medicament, providing a nose piece adapted for nasal delivery of the medicament, the nose piece being fixable to the body at an acute angle to the elongate body, a first portion of the nose piece defining an outlet for discharging a medicament from the actuator, and fixing means for fixing the nose piece to the body, wherein the nose piece is adapted and the body is adapted so that when the nose piece is fixed to the body the nose piece and body cooperate to define a fluid communication pathway from the canister opening to the outlet.

Throughout this specification, the word “inhaler” means a device for delivery of a medicament in fluid (or powder) and does not imply that the device requires inhalation on the part of the patient during delivery. It is known that a medicament may be delivered successfully to the nasal passages by an inhaler without the need for the patient to inhale.

So that the present specification may be more completely understood, reference is made, by way of example only, to the accompanying drawings in which:

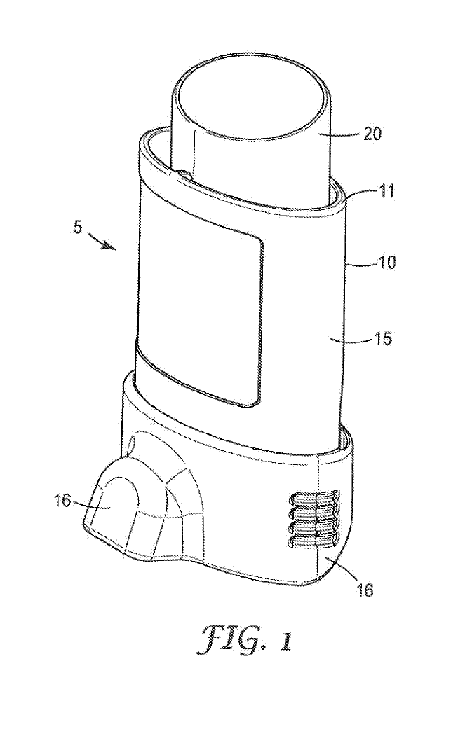

FIG. 1 is an isometric front view of an embodiment of an inhaler from a frontal angle.

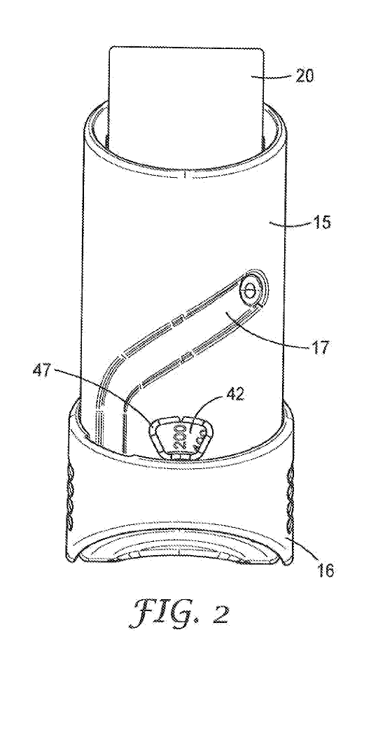

FIG. 2 is a rear view of the inhaler of FIG. 1.

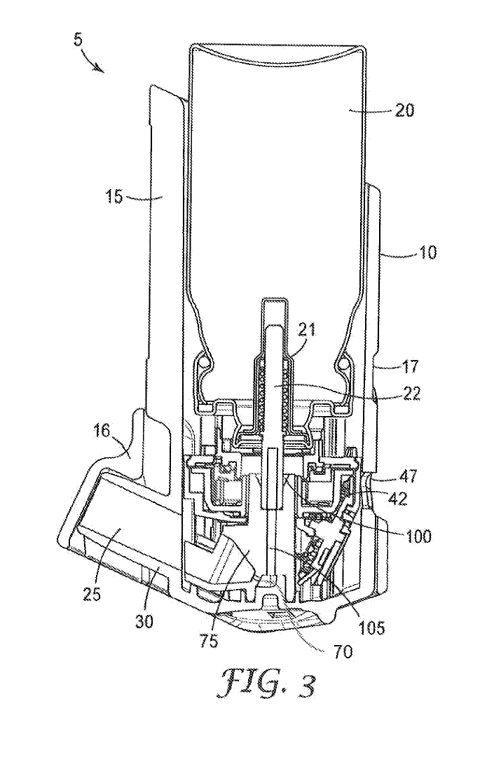

FIG. 3 illustrates a vertical section through an inhaler as disclosed in GB 1308679.8.

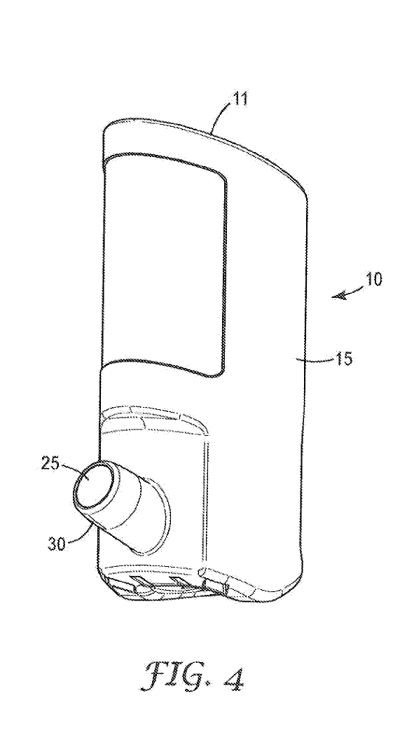

FIG. 4 is an angled isometric view of the actuator of the inhaler of FIGS. 1 and 2.

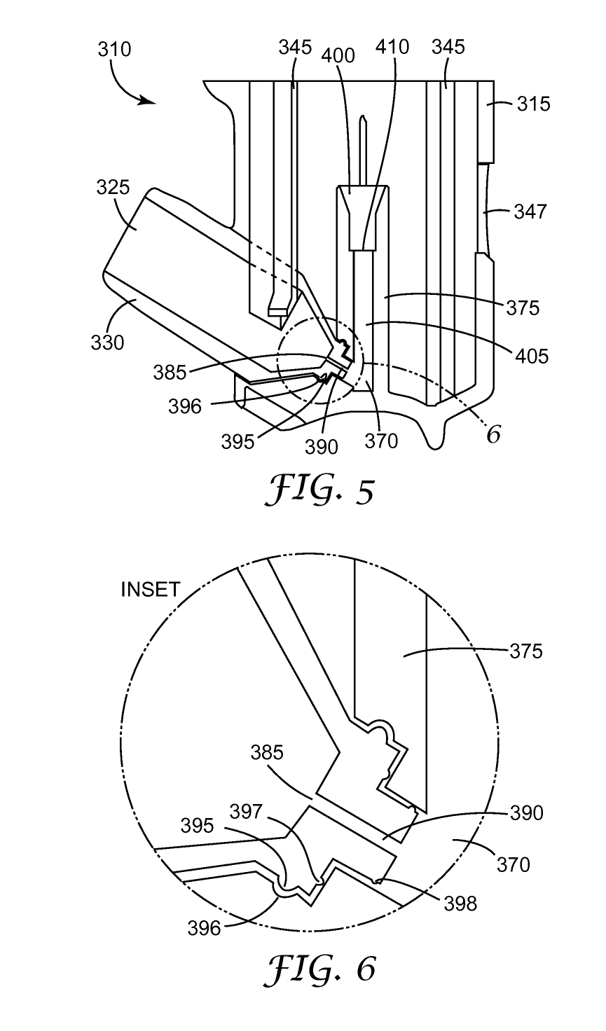

FIG. 5 is a vertical section through the lower part of an embodiment of an actuator according to the present invention.

FIG. 6 is an inset of the circled portion of FIG. 5.

FIG. 1 illustrates, in frontal isometric view, a pressurised metered dose inhaler 5 comprising a canister 20 and an actuator 10. The canister 20 is pressurised and holds medicament for delivery via the actuator 10. The actuator 10 has a generally elongate actuator body 15 that acts as a housing for the canister 20. The canister 20 is inserted into the canister opening 11 at the top portion of the actuator 10. The inhaler 5 is a nasal inhaler, having a nose piece (see FIG. 4) covered by a cap 16.

FIG. 2 shows, in a rear view, the actuator body 15, the actuator cap 16, and the canister 20. The body 15 has a cap track 17 arranged to guide the cap 16 from a closed position in which the cap 16 covers the nose piece (the position as shown in FIGS. 1 and 2) to an open position (not shown) in which the nose piece 30 is uncovered. The body 15 has a viewing window 47 through which the display of a dose indicator 42 is visible.

FIG. 3 shows a vertical section through an inhaler 5 as disclosed in GB 1308679.8. The actuator 10 comprises a body 15 having a stem post 75. The stem post 75 has a stem socket 100 for receiving the canister valve stem 22 of the canister metering valve 21. In use, the patient would displace cap 16 from the nose piece 30, insert the nose piece 30 into a nostril and exert pressure on the top of the canister 20. This moves the canister 20 into the body 15 of the actuator and presses the canister valve stem 22 against the stem post 75, resulting in the canister metering valve 21 opening and releasing a metered dose of medicament into an expansion chamber 105 within the stem post 75. The expansion chamber 105 is in fluid communication, via a transition chamber 70, with a delivery passage 25 in the body 15 so that the medicament is delivered through the delivery passage 25 and out of the nose piece 30 into the patient's nostril. A dose indicator 42 is situated at the lower rear portion of the body 15 so that its indicia are visible through the indicator window 47. The dose indicator 42 is so arranged that movement of the canister 20 in use indexes the dose indicator 42.

FIG. 4 shows an actuator 10 with the cap 16 and canister 20 removed. The nose piece 30 is angled upwardly at an acute angle with respect to the long axis of the body 15 for convenient insertion into the nostril of a patient. The nose piece 30 has a delivery passage 25 through which the medicament is delivered.

FIG. 5 shows a section through the lower portion of the actuator 310 of the present invention. Many aspects of the actuator of FIG. 5 are similar to that of FIG. 3. However, FIG. 5 illustrates an actuator 310 having an elongate body 315 and a separate nose piece 330. The nose piece 330 and elongate body 315 are each unitary mouldings. The nose piece 330 and elongate body 315 are moulded as separate pieces that may be fixed together. In FIG. 5, the dose indicator 42 and canister 20 (shown in FIG. 3) are not shown, although an indicator window 347 for a dose indicator is shown. FIG. 5 shows ribs 345 that locate the canister (not shown in FIG. 5) in the correct position in the elongate body 315 of the actuator 310. With the nose piece 330 fixed with the body 315, the delivery passage in the nose piece 330 is able to receive doses of medicament from the canister through an orifice outlet 385. The orifice outlet 385 is in fluid communication through a jet portion 390 with a transition chamber 370 forming part of the expansion chamber 405. The jet portion 390 is of predetermined width and predetermined length and may be in the form of a cylindrical tube of diameter 0.1 to 1.5 mm and of length 0.05 to 5 mm, the length usually being 0.4 mm to 3 mm. The delivery passage defines an outlet 325 for discharging a medicament from the actuator. The separate nose piece 330 is produced as a separate piece that is fixable in the body 315 by means of a press fit sealing ring 395 on the nose piece 330 in a press fit sealing groove 396 on the body 315. Further detail on how the nose piece 330 is fixed in the body is shown in the expanded inset, FIG. 6. Circumferential crush beads 397, 398 are provided on two surfaces of the nosepiece to facilitate the sealing of the nose piece with the transition chamber.

In use, the stem socket 400 accepts the tip of a valve stem of the canister (not shown in FIG. 5), the end of the valve stem of the canister being supported on a stem ledge 410. The stem ledge 410 and/or the expansion chamber 405 may be generally of any cross sectional shape (e.g. oval, rectangular, D shaped), but preferably the stem ledge is annular, and the expansion chamber cylindrical. A metered dose of medicament is delivered from the valve of the canister through the expansion chamber 405 of the stem post 375, into the transition chamber 370, and out of the orifice outlet 385 via the jet portion 390. From the orifice outlet 385, the dose is delivered through the delivery passage and then discharged from the outlet 325 and hence from the nose piece 330 into the nostril of the patient.

It is to be understood that the specification is not limited to the embodiments described above and that various modifications can be made without departing from the principles or concepts of the specification.

Actuators and inhalers according to the specification may include any feature described herein separately or in combination with any other feature(s), if necessary with appropriate modification of other features, as would be readily apparent to the skilled person.

1. An actuator for a nasal inhaler, the actuator comprising an elongate body comprising a canister opening for insertion of a canister for supplying a medicament,
a nose piece adapted for nasal delivery of the medicament, the nose piece being fixable to the body at an acute angle to the elongate body, a first portion of the nose piece defining an outlet for discharging a medicament from the actuator, and
fixing means for fixing the nose piece to the body,
wherein the nose piece is adapted and the body is adapted so that when the nose piece is fixed to the body the nose piece and body cooperate to define a fluid communication pathway from the canister opening to the outlet.

2. An actuator as claimed in claim 1, further comprising a second portion of the nose piece defining an orifice through which medicament may pass from the body to the nose piece.

3. An actuator as claimed in either claim 1 or claim 2, wherein the canister has a valve stem and wherein the actuator further comprises a stem socket adapted for receiving the valve stem of the canister.

4. An actuator as claimed in claim 3, wherein the stem socket is comprised in a stem post.

5. An actuator as claimed in any one of claims 2 to 4, wherein the orifice has a jet portion of predetermined width and predetermined length.

6. An actuator as claimed in any one of the preceding claims wherein the fixing means comprises at least one seal feature.

7. An actuator as claimed in claim 6, wherein the at least one seal comprises a ring and groove press fit seal.

8. An actuator as claimed in any one of the preceding claims, wherein the nose piece is a unitary moulding.

9. An actuator as claimed in any one of the preceding claims, wherein the elongate body is a unitary moulding.

10. An actuator as claimed in any one of the preceding claims, wherein the actuator further comprises a window, preferably an indicator and/or dose counter viewing window.

11. An actuator as claimed in any one of the preceding claims, further comprising supporting means for supporting a dose counter.

12. An actuator as claimed in any one of the preceding claims, wherein the nosepiece is angled upwardly.

13. A nasal inhaler for delivery of a medicament, the nasal inhaler comprising an actuator as claimed in any one of the preceding claims and a canister.
